(19) United States
(12) Patent Application Publication
(10) Pub. No.: US 2017/0151144 A1
(43) Pub. Date: Jun. 1, 2017

(54) HIGH-PERFORMANCE HAIR TREATMENT AGENTS HAVING ANTI-WASHOUT EFFECT

(71) Applicant: Henkel AG & Co. KGaA, Duesseldorf (DE)

(72) Inventors: Volker Scheunemann, Lueneburg (DE); Erik Schulze zur Wiesche, Hamburg (DE)

(73) Assignee: Henkel AG & Co. KGaA, Duesseldorf (DE)

(21) Appl. No.: 15/360,285

(22) Filed: Nov. 23, 2016

(30) Foreign Application Priority Data
Dec. 1, 2015 (DE) 10 2015 223 829.6

(51) Int. Cl. A61K 8/43; A61K 8/41; A61K 8/23; A61Q 5/02; A61K 8/365; A61K 8/36; A61Q 5/12; A61K 8/34; A61K 8/362

(52) U.S. Cl. CPC A61K 8/43; A61K 8/342; A61K 8/416; A61K 8/23; A61K 8/362; A61K 8/365; A61K 8/36; A61Q 5/12; A61Q 5/02

(57) ABSTRACT

The disclosure relates to hair treatment agents comprising at least one fatty alcohol, at least one cationic surfactant and at least one divalent or trivalent metal salt. At least one organic acid and ethyl lauroyl arginate reduce or prevent dye from being washed out of colored hair.

### CROSS-REFERENCE TO RELATED APPLICATION

This application claims priority to German Patent Application No. 10 2015 223 829.6, filed Dec. 1, 2015, which is incorporated herein by reference in its entirety.

### TECHNICAL FIELD

This disclosure relates to hair treatment agents, in particular to shampoos and so-called conditioners, comprising an active ingredient combination for gentle and effective hair care.

### BACKGROUND

Due in no small part to severe stressing of the hair, for example as a result of coloring or permanent-wave treatment, but also as a result of cleaning the hair with shampoos and due to environmental pollution, the significance of care products having a preferably long-lasting effect is on the rise. Such care agents influence the natural structure and properties of the hair. Subsequent to such care treatments, for example, the wet and dry combability of the hair, and the hold and volume of the hair may be optimized, or the hair may be protected from increased split ends.

It has therefore been customary for some time to apply a special after-treatment to hair. To this end, the hair is treated with special active ingredients, usually in the form of a rinse conditioner, such as quaternary ammonium salts or special polymers. Depending on the formulation, this treatment improves the combability, hold and volume of the hair and reduces split ends.

Likewise, multifunctional cosmetic products are known in the prior art. These include in particular what are known as “2-in-1” shampoos, which not only clean, but also condition the hair. These products are valued highly by the consumer since the product performance thereof eliminates at least one method step, for example conditioning using a traditional hair rinse conditioner.

In addition, products used to change the natural hair color play a prominent role in hair cosmetics. A distinction is made between permanent, semi-permanent and temporary coloring systems, which are based on chemical and/or natural dyes. Hair colors artificially created by permanent, semi-permanent or temporary coloring systems, however, have the disadvantage that these may undesirably change, for example during or after cleaning the hair.

An “undesirable change” shall be understood to mean fading or bleeding and the loss of luster of the hue of the hair achieved by the particular coloring process. These changes may be further amplified by environmental factors and/or sunlight.

The use of divalent metal salts in hair coloring agents to improve the longevity, and thus the fastness, of the coloration is known from EP 2438900 A1.

A continued need exists for active ingredients or active ingredient combinations for hair treatment agents having good nourishing properties, which additionally strengthen the adherence of dyes to the hair fibers and thereby preserve the fastness of the artificially created hair color, and for further development of hair treatment agents in this regard.

### BRIEF SUMMARY

Hair treatment agents and methods for treating hair using the hair treatment agents are provided. In accordance with an exemplary embodiment, a hair treatment agent comprises at least one fatty alcohol, at least one cationic surfactant, at least one divalent or trivalent metal salt, at least one organic acid, and ethyl lauroyl arginate.

In accordance with another exemplary embodiment, a method for treating hair comprises the steps of applying to dry or moist hair a hair treatment agent comprising at least one fatty alcohol, at least one cationic surfactant, at least one divalent or trivalent metal salt, at least one organic acid, and ethyl lauroyl arginate. The hair treatment agent is rinsed from the hair after it is left on the hair for a period of about 30 to 300 seconds.

### DETAILED DESCRIPTION

The following detailed description is merely exemplary in nature and is not intended to limit the invention or the application and uses of the invention. Furthermore, there is no intention to be bound by any theory presented in the preceding background or the following detailed description.

It has now been found that a combination of certain ingredients has a particularly positive effect on colored hair treated therewith and the hair follicles.
A first embodiment of the present invention is hair treatment agents, comprising:
a) at least one fatty alcohol;
b) at least one cationic surfactant;
c) at least one divalent or trivalent metal salt.
d) at least one organic acid;
e) ethyl lauroyl arginate.

Hair treatment agents as contemplated herein are, for example, hair shampoos, hair conditioners, conditioning shampoos, hair sprays, hair rinse conditioners, deep hair conditioners, hair packs, hair tonics, permanent wave fixation solutions, hair coloring shampoos, hair coloring agents, hair setting lotions, hair style setting agents, hair styling preparations, blow drying lotions, mousse, hair gels, hair waxes or the combinations thereof. With respect to the fact that men in particular often shy away from the use of several different products and/or multiple application steps, agents that men use anyhow are preferred. Preferred agents are therefore shampoos, conditioning agents or hair tonics.

The hair treatment agents comprise at least one fatty alcohol. Fatty alcohols are aliphatic, long-chain, monohydric primary alcohols comprising hydrocarbon groups that have 6 to 30, and preferably 6 to 22 carbon atoms. The hydrocarbon groups may be saturated, monounsaturated or polyunsaturated. Preferred fatty alcohols that can preferably be used within the scope of the present invention are selected from 1-hexanol, 1-heptanol, 1-octanol, 1-decanol, 1-dodecanol (lauryl alcohol), 1-tetradecanol (myristyl alcohol), 1-hexadecanol (cetyl alcohol), 1-heptadecanol (margaryl alcohol), 1-octadecanol (stearyl alcohol), 1-eisosanol (arachidyl alcohol), 1-docosanol (behenyl alcohol), 1-tetracosanol (lignoceryl alcohol), 1-hexacosanol (ceryl alcohol), 1-octacosanol (montanyl alcohol), 1-triacontanol (melissyl alcohol), cis-9-hexadecen-1-ol (palmitoleyl alcohol), cis-9-octadecen-1-ol (oleyl alcohol), trans-9-octadecen-1-ol (elaidyl alcohol), cis-11-octadecen-1-ol, 6,9,12-octadecatrien-1-ol (γ-linolenyl alcohol) and the mixtures thereof.

Particularly preferred hair treatment agents are characterized by comprising, based on the weight thereof, about 0.1 to about 20 wt. %, especially about 0.5 to about 15 wt. %, more preferably about 1 to about 10 wt. %, and in particular about 2 to about 8 wt. % fatty alcohol(s) of formula (II)

H₃C—(CH₂)ₖ—CH₂—OH (II)

in which k denotes integers from 4 to 28, especially from 6 to 24, more preferably from 8 to 22, and in particular 10, 12, 14, 16, 18 or 20.

Particularly preferred hair treatment agents as contemplated herein comprise, based on the weight thereof, about 0.1 to about 20 wt. %, especially about 0.5 to about 15 wt. %, more preferably about 1 to about 10 wt. %, and in particular about 2 to about 8 wt. % alcohol(s) from the group consisting of 1-dodecanol (lauryl alcohol), 1-hexadecanol (cetyl alcohol), 1-octadecanol (stearyl alcohol) and 1-docosanol (behenyl alcohol), wherein the amounts refer to the total amount of the aforementioned fatty alcohols in the composition.

Especially particularly preferred hair treatment agents as contemplated herein comprise, based on the weight thereof, about 0.1 to about 20 wt. %, especially about 0.5 to about 15 wt. %, more preferably about 1 to about 10 wt. %, and in particular about 2 to about 8 wt. % alcohol(s) from the group consisting of 1-hexadecanol (cetyl alcohol) and 1-octadecanol (stearyl alcohol), wherein the amounts refer to the total amount of the aforementioned fatty alcohols in the composition.

The hair treatment agents comprise at least one cationic surfactant. The cationic surfactant(s) is or are preferably selected from the group of the quaternary ammonium compounds and/or of the amidoamines, wherein preferred cationic surfactant(s) is/are selected from
alkyl trimethylammonium chlorides having preferably 10 to 18 carbon atoms in the alkyl group and/or
dialkyldimethylammonium chlorides having preferably 10 to 18 carbon atoms in the alkyl group and/or
trialkylmethylammonium chlorides having preferably 10 to 18 carbon atoms in the alkyl group and/or
cetyltrimethylammonium chloride and/or
stearyltrimethylammonium chloride and/or
behenyltrimethylammonium chloride and/or
distearyldimethylammonium chloride and/or
lauryldimethylammonium chloride and/or
lauryldimethyl benzylammonium chloride and/or
tricetylmethylammonium chloride
Quaternium-27 and/or
Quaternium-83.

Preferred hair treatment agents contemplated herein comprise, based on the weight thereof, about 0.05 to about 20 wt. %, especially about 0.1 to about 10 wt. %, more preferably about 0.25 to about 8 wt. %, and in particular about 0.5 to about 7 wt. % cationic surfactant(s).

Particularly preferred cationic surfactants are selected from compounds of the following formula (I)

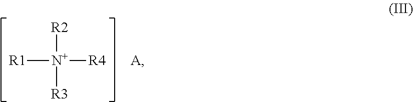

in which no more than three groups R1 to R4, independently of one another, denote a saturated or unsaturated, branched or unbranched alkyl group having 1 to 4 carbon atoms, at least one group R1 to R4 denotes a saturated or unsaturated, branched or unbranched alkyl chain having 8 to 30 carbon atoms, and A denotes a physiologically compatible organic or inorganic anion,

In preferred compounds according to formula (I),
two or three groups R1 to R4 denote a methyl group or an ethyl group;
one or two groups R1 to R4 denote a saturated or unsaturated, branched or unbranched alkyl chain having 14 to 26 carbon atoms; and
A denotes a halide ion, a sulfate ion of general formula RSO₃⁻, where R has the meaning of saturated or unsaturated alkyl groups having 1 to 4 carbon atoms, or an anionic group of an organic acid such as maleic acid, fumaric acid, oxalic acid, tartaric acid, citric acid, lactic acid or acetic acid.

More preferred are compounds according to formula (III), in which
three groups R1 to R4 denote a methyl group;
a group R1 to R4 denotes a cetyl, palmityl, stearyl, arachidyl or behenyl group; and
A denotes a chloride or a methosulfate ion.

The at least one compound according to formula (I) is particularly preferably selected from cetyltrimethylammonium chloride, cetyltrimethylammonium methosulfate, behentrimethylammonium chloride and/or behentrimethylammonium methosulfate. These compounds can be used individually or in the combination thereof in the agent contemplated herein, wherein the total amount of compounds of formula (I) in the agent is preferably no more than about 10 wt. %, and wherein the quantity information refers to the total weight of the agent contemplated herein.

In particular, an agent as contemplated herein preferably comprises behentrimethylammonium chloride as the cationic surfactant. Hair treatment agents as contemplated herein that are preferred here are those that, based on the weight thereof, comprise about 0.05 to about 20 wt. %, especially about 0.1 to about 10 wt. %, more preferably about 0.25 to about 8 wt. %, and in particular about 0.5 to about 7 wt. % behenyltrimethylammonium chloride.

The hair treatment agents can also comprise at least one esterquat as the cationic surfactant. “Esterquats” as used herein shall preferably be understood to mean compounds of the following formula (IV)

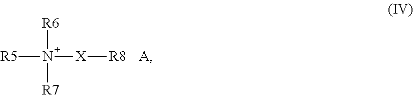

in which
the groups R5, R6 and R7, each independently of one another, may be the same or different and have the following meanings:
a saturated or unsaturated, branched or unbranched alkyl group having 1 to 4 carbon atoms, which may comprise at least one hydroxyl group; or
a saturated or unsaturated, branched or unbranched, or a cyclic saturated or unsaturated, alkyl group having 6 to 30 carbon atoms, which may comprise at least one hydroxyl group; or
an aryl or alkylaryl group, such as phenyl or benzyl; or
(—X—R8), with the proviso that no more than 2 of the groups R5, R6 or R7 can denote (—X—R8), wherein
   X has the following meaning:
—(CH2)n- where n=1 to 20, especially n=1 to 10, and particularly preferably n=1 to 5; or
—(CH₂—CHR9-O)ₙ—, where n=1 to 200, especially 1 to 100, more preferably 1 to 50, and particularly preferably 1 to 20, and where R9 has the meaning of hydrogen, methyl or ethyl; or
a hydroxyalkylene group having one to four carbon atoms, which may be branched or unbranched, and which comprises at least one and no more than 3 hydroxyl groups; and wherein
   R8 has the following meaning:
R10-O—CO—, where R10 is a saturated or unsaturated, branched and unbranched, or a cyclic saturated or unsaturated, alkyl group having 6 to 30 carbon atoms, which may comprise at least one hydroxyl group, and which optionally may furthermore be oxethylated with 1 to 100 ethylene oxide units and/or 1 to 100 propylene oxide units; or
R11-CO—, where R11 is a saturated or unsaturated, branched and unbranched, or a cyclic saturated or unsaturated, alkyl group having 6 to 30 carbon atoms, which may comprise at least one hydroxyl group, and which optionally may furthermore be oxethylated with 1 to 100 ethylene oxide units and/or 1 to 100 propylene oxide units, and
   in which A denotes a physiologically compatible organic or inorganic anion; preferably, one of the groups R5, R6 or R7 denotes the group (—X—R8), R8 denotes a non-ethoxylated fatty acid group, such as a palmitic, stearic, arachidic or behenic acid group, in particular a stearic acid group, and A denotes a halide ion, a sulfate ion of the general formula RSO₃⁻, where R has the meaning of saturated or unsaturated alkyl groups having 1 to 4 carbon atoms, or an anionic group of an organic acid such as maleic acid, fumaric acid, oxalic acid, tartaric acid, citric acid, lactic acid or acetic acid, and in particular a chloride ion or a methosulfate ion.

The esterquats suitable for the agents contemplated herein are preferably selected from at least one of the products sold under the trade names Rewoquat®, Stepantex®, Dehyquart®, Armocare® and Akypoquat®. Specific examples of esterquats that are particularly suited herein are the products Armocare® VGH-70, Dehyquart® F-75, Dehyquart® C-4046, Dehyquart® L80, Dehyquart® F-30, Dehyquart® AU-35, Rewoquat® WE18, Rewoquat® WE38 DPG, Stepantex® VS 90 and Akypoquat® 131.

Particularly preferred are agents contemplated herein that comprise at least one of the compounds known by the INCI names Distearoylethyl Hydroxyethylmonium Methosulfate and Distearoylethyl Hydroxyethylmonium Chloride as esterquats. In particular, Distearoylethyl Hydroxyethylmonium Methosulfate is preferred, which may be present in the agents contemplated herein in a preferred amount of about 0.1 to about 10 wt. %, more preferably about 0.5 to about 8 wt. %, particularly preferably about 0.75 to about 6 wt. %, and in particular about 1 to about 5 wt. %, wherein the quantity information refers to the total weight of the agent contemplated herein.

The esterquat or esterquats can be added to the agents contemplated herein either individually or as a mixture with other active nourishing agents. To enable better handling and processing, it may be advantageous to add the esterquat or esterquats, and in particular Distearoylethyl Hydroxyethylmonium Methosulfate, to the agents contemplated herein in the form of an active ingredient mixture. A particularly suitable example of such an active ingredient mixture is available from BASF, for example, under the trade name Dehyquart® F 75 (Distearoylethyl Hydroxyethylmonium Methosulfate and Cetearyl Alcohol).

In an exemplary embodiment, the hair treatment agents comprise at least one divalent or trivalent metal salt. To achieve optimal action, it is advantageous if the metal salts are present in dissolved form in the agents contemplated herein. In a preferred embodiment, the hair cleaning and care agents contemplated herein thus contain water-soluble divalent or trivalent metal salts. The term “water-soluble” shall be understood to mean that at least 1 g of the particular salt can be completely dissolved in 1 l water at 20° C.

Suitable divalent or trivalent metal salts may be selected from divalent or trivalent organic and/or inorganic salts.

Particularly suitable cations within these salts can preferably be selected from alkaline earth metal cations, as well as copper, zinc, iron(II), iron(III) and/or aluminum cations. Alkaline earth metal cations are especially particularly preferred, and in particular calcium and magnesium cations are preferred. Particularly suitable organic anions within these salts can particularly be selected from acetate, lactate, succinate, citrate, tartrate, malate, maleate, oxalate and/or glycolate ions. Especially particularly preferred are acetate, lactate and/or citrate salts comprising the aforementioned cations. In particular, preferred organic salts are calcium lactate, calcium citrate, calcium acetate, magnesium lactate, magnesium citrate and/or magnesium acetate. Particularly suitable inorganic anions within these salts may be selected from halide, sulfate, phosphate and/or carbonate ions. Sulfate and/or halide ions are especially particularly preferred, such as chloride and bromide ions. In particular, preferred inorganic salts are calcium chloride, calcium sulfate, magnesium chloride and/or magnesium sulfate.

The percentage of weight of the at least one divalent or trivalent metal salt based on the total weight of the hair treatment agent contemplated herein is preferably about 0.01 to about 10 wt. %, especially about 0.1 to about 7.5 wt. %, more preferably about 0.2 to about 5 wt. %, and in particular about 0.3 to about 3 wt. %.

Preferred hair treatment agents contemplated herein comprise, based on the weight thereof, about 0.01 to about 10 wt. %, especially about 0.1 to about 7.5 wt. %, more preferably about 0.2 to about 5 wt. %, and in particular about 0.3 to about 3 wt. % of at least one divalent or trivalent metal salt from the group of the organic or inorganic copper, zinc, iron(II), calcium, magnesium, iron(III) and/or aluminum salts.

Within this embodiment, the water-soluble salts are particularly preferred. Especially particularly preferred within this embodiment are calcium lactate, calcium citrate, calcium acetate, magnesium lactate, magnesium citrate, magnesium acetate, calcium halide, calcium hydroxide, magnesium halide and/or magnesium hydroxide.

In an exemplary embodiment, the hair treatment agents contemplated herein comprise at least one organic acid. Typical representatives for organic acids are aliphatic monocarboxylic and dicarboxylic acids, such as acetic acid, propionic acid, oxalic acid, and 1,3-propanedioic acid, as well as aromatic carboxylic acids, such as benzoic acid. Further organic acids are, for example, hydroxycarboxylic acids such as glycolic acid, citric acid, tartaric acid, malic acid and lactic acid. Unsaturated monocarboxylic or dicarboxylic acids, such as fumaric acid, or α-keto carboxylic acids, such as pyruvic acid (2-oxopropionic acid) are also contemplated herein.

Preferred hair treatment agents contemplated herein comprise, based on the weight thereof, about 0.001 to about 10 wt. %, especially about 0.01 to about 5 wt. %, more preferably about 0.05 to about 3 wt. %, and in particular about 0.1 to about 2 wt. % organic acid(s).

Particularly preferred hair treatment agents comprise, based on the weight thereof, about 0.001 to about 10 wt. %, especially about 0.01 to about 5 wt. %, more preferably about 0.05 to about 3 wt. %, and in particular about 0.1 to about 2 wt. % organic acid(s) from the group consisting of formic acid, acetic acid, propionic acid, butyric acid, isobutyric acid, valeric acid, isovaleric acid, pivalic acid, oxalic acid, malonic acid, succinic acid, glutaric acid, glyceric acid, glyoxylic acid, adipic acid, pimelic acid, suberic acid, azelaic acid, sebacic acid, propiolic acid, crotonic acid, isocrotonic acid, elaidic acid, maleic acid, fumaric acid, muconic acid, citraconic acid, mesaconic acid, camphoric acid, benzoic acid, o,m,p-phthalic acid, naphthoic acid, toluoyl acid, hydratropic acid, atropic acid, cinnamic acid, isonicotinic acid, nicotinic acid, bicarbamic acid, 4,4′-dicyano-6,6′-binicotinic acid, 8-carbamoyloctanoic acid, 1,2,4-pentanetricarboxylic acid, 2-pyrrole carboxylic acid, 1,2,4,6,7-napthalene pentaacetic acid, malonaldehyde acid, 4-hydroxyphthalimide acid, 1-pyrazole carboxylic acid, gallic acid or propanetricarboxylic acid, glycolic acid, lactic acid, malic acid, tartaric acid or citric acid.

Several representatives among the aforementioned acids are particularly preferred since these additionally impart a pleasant shine to the hair treated with the agents contemplated herein. Exceptionally preferred hair treatment agents comprise, based on the weight thereof, about 0.001 to about 10 wt. %, especially about 0.01 to about 5 wt. %, more preferably about 0.05 to about 3 wt. %, and in particular about 0.1 to about 2 wt. % organic acid(s) from the group consisting of formic acid, oxalic acid, maleic acid, lactic acid, tartaric acid or citric acid.

In an exemplary embodiment, the agents contemplated herein comprise ethyl lauroyl arginate (ELA or LAE)

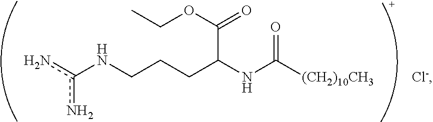

which is also referred to as ethyl-N^{alpha}-dodecanoyl-L-arginate hydrochloride. LAE is commercially available and considered to be safe, also for use in foods.

Preferred hair treatment agents contemplated herein comprise, based on the weight thereof, about 0.001 to about 2 wt. %, especially about 0.01 to about 1 wt. %, more preferably about 0.025 to about 0.6 wt. %, and in particular about 0.05 to about 0.4 wt. % ethyl lauroyl arginate HCl.

The hair treatment agents preferably comprise the above-described active ingredients in a cosmetically acceptable carrier. Within the scope of the invention, this is preferably understood to mean an aqueous or aqueous-alcoholic carrier. The cosmetic carrier preferably contains at least about 50 wt. %, more preferably at least about 60 wt. %, particularly preferably at least about 70 wt. %, and in particular preferably at least about 75 wt. % water. Furthermore, the cosmetic carrier can comprise about 0.01 to about 40 wt. %, preferably about 0.05 to about 30 wt. %, and in particular about 0.1 to about 20 wt. % of at least one alcohol. Suitable alcohols are, for example, ethanol, ethyl diglycol, 1-propanol, 2-propanol, isopropanol, 1,2-propylene glycol, glycerol, diglycerol, triglycerol, 1-butanol, 2-butanol, 1,2-butanediol, 1,3-butanediol, 1-pentanol, 2-pentanol, 1,2-pentanediol, 1,5-pentanediol, 1-hexanol, 2-hexanol, 1,2-hexanediol, 1,6-hexanediol, polyethylene glycols, sorbitol, sorbitan, benzyl alcohol, or mixtures of these alcohols. The water-soluble alcohols are particularly preferred. In particular, ethanol, 1,2-propylene glycol, glycerol, benzyl alcohol and mixtures of these alcohols are preferred.

For excellent skin (scalp) tolerability of the hair treatment agents contemplated herein, it is advantageous if these have a slightly acidic pH value. It was found that the agents contemplated herein offer particularly good skin tolerability and mildness in a pH range from about 4.2 to about 5.8.

In a first preferred embodiment, the hair treatment agents contemplated herein thus preferably have a pH value in the range of about 4.2 to about 5.8, more preferably of about 4.25 to about 5.6, particularly preferably of about 4.3 to about 5.5, exceptionally preferably of about 4.35 to about 5.4, and in particular preferably of about 4.4 to about 5.3.

In an exemplary embodiment, the hair treatment agents contemplated herein can comprise silicone(s).

Preferred agents contemplated herein are characterized by comprising at least one silicone, which is selected from:
(i) polyalkylsiloxanes, polyarylsiloxanes, polyalkylarylsiloxanes, which are volatile or non-volatile, straight-chain, branched or cyclic, cross-linked or not cross-linked;
(ii) polysiloxanes containing, in the general structure thereof, one or more organofunctional groups, which are selected from:
   a) substituted or unsubstituted aminated groups;
   b) (per)fluorinated groups;
   c) thiol groups;
   d) carboxylate groups;
   e) hydroxylated groups;
   f) alkoxylated groups;
   g) acyloxyalkyl groups;
   h) amphoteric groups;
   i) bisulfite groups;
   j) hydroxyacylamino groups;
   k) carboxy groups;
   l) sulfonic acid groups; and
   m) sulfate or thiosulfate groups;
(iii) linear polysiloxane(A)/polyoxyalkylene(B) block copolymers of the (A-B)ₙ type where n>3;
(iv) grafted silicone polymers having a non-silicone-containing, organic skeleton, which are composed of an organic main chain formed of organic monomers that do not contain any silicone, onto which at least one polysiloxane macromer was grafted in the chain and optionally on at least one chain end;
(v) grafted silicone polymers having a polysiloxane skeleton onto which non-silicone-containing, organic monomers were grafted that include a polysiloxane main chain, onto which at least one organic macromer that does not contain silicone was grafted in the chain and optionally on at least one of the ends thereof;
   or the mixtures thereof.

Preferred hair treatment agents contemplated herein are characterized by comprising, based on the weight thereof, about 0.01 to about 20 wt. %, especially about 0.1 to about 10 wt. %, more preferably about 0.5 to about 7.5 wt. %, and in particular about 1 to about 5 wt. % silicone(s).

Preferred silicones are described hereafter.

Particularly preferred agents contemplated herein are characterized by comprising at least one silicone of formula Si-I

(CH₃)₃Si—[O—Si(CH₃)₂]ₓ—O—Si(CH₃)₃ (Si-I),

in which x denotes a number from 0 to 100, preferably from 0 to 50, more preferably from 0 to 20, and in particular 0 to 10.

According to INCI nomenclature, these silicones are referred to as DIMETHICONE. Within the scope of the present invention, the silicone of formula Si-I used is preferably one of the following compounds:

(CH₃)₃Si—O—Si(CH₃)₃, (CH₃)₃Si—O—(CH₃)₂Si—O—Si(CH₃)₃, (CH₃)₃Si—[O—(CH₃)₂Si]₂—O—Si(CH₃)₃, (CH₃)₃Si—[O—(CH₃)₂Si]₃—O—Si(CH₃)₃, (CH₃)₃Si—[O—(CH₃)₂Si]₄—O—Si(CH₃)₃, (CH₃)₃Si—[O—(CH₃)₂Si]₅—O—Si(CH₃)₃, (CH₃)₃Si—[O—(CH₃)₂Si]₆—O—Si(CH₃)₃, (CH₃)₃Si—[O—(CH₃)₂Si]₇—O—Si(CH₃)₃, (CH₃)₃Si—[O—(CH₃)₂Si]₈—O—Si(CH₃)₃, (CH₃)₃Si—[O—(CH₃)₂Si]₉—O—Si(CH₃)₃, (CH₃)₃Si—[O—(CH₃)₂Si]₁₀—O—Si(CH₃)₃, (CH₃)₃Si—[O—(CH₃)₂Si]₁₁—O—Si(CH₃)₃, (CH₃)₃Si—[O—(CH₃)₂Si]₁₂—O—Si(CH₃)₃, (CH₃)₃Si—[O—(CH₃)₂Si]₁₃—O—Si(CH₃)₃, (CH₃)₃Si—[O—(CH₃)₂Si]₁₄—O—Si(CH₃)₃, (CH₃)₃Si—[O—(CH₃)₂Si]₁₅—O—Si(CH₃)₃, (CH₃)₃Si—[O—(CH₃)₂Si]₁₆—O—Si(CH₃)₃, (CH₃)₃Si—[O—(CH₃)₂Si]₁₇—O—Si(CH₃)₃, (CH₃)₃Si—[O—(CH₃)₂Si]₁₈—O—Si(CH₃)₃, (CH₃)₃Si—[O—(CH₃)₂Si]₁₉—O—Si(CH₃)₃, (CH₃)₃Si—[O—(CH₃)₂Si]₂₀—O—Si(CH₃)₃, wherein (CH₃)₃Si—O—Si(CH₃)₃, (CH₃)₃Si—O—(CH₃)₂Si—O—Si(CH₃)₃ and/or (CH₃)₃Si—[O—(CH₃)₂Si]₂—O—Si(CH₃)₃ are particularly preferred. It is also possible, of course, for mixtures of the above-mentioned silicones to be present in the agents contemplated herein. Preferred silicones that can be used herein have viscosities of about 0.2 to about 2 mm²s⁻¹ at 20° C., wherein silicones having viscosities of about 0.5 to about 1 mm²s⁻¹ are particularly preferred.

Particularly preferred agents contemplated herein comprise one or more aminofunctional silicones. Such silicones can be described, for example, by the following formula

M(RₐQ_{b}SiO_{(4-a-b))/2)x}(R_{c}SiO_{(4-c))/2)y}M

wherein, in the above formula, R is a hydrocarbon or a hydrocarbon group having 1 to approximately 6 carbon atoms, Q is a polar group of the general formula —R¹HZ, where R¹ is a divalent, linking group, which is bound to hydrogen and the group Z, composed of carbon and hydrogen atoms, carbon, hydrogen and oxygen atoms, or carbon, hydrogen and nitrogen atoms, and Z is an organic, aminofunctional group comprising at least one aminofunctional group; “a” takes on values in the range of approximately 0 to approximately 2, “b” takes on values in the range of approximately 1 to approximately 3, “a”+“b” is smaller than or equal to 3, and “c” is a number in the range of approximately 1 to approximately 3, and x is a number in the range of 1 to approximately 2000, especially of approximately 3 to approximately 50, and more preferably of approximately 3 to approximately 25, and y is a number in the range of approximately 20 to approximately 10,000, especially of approximately 125 to approximately 10,000, and most preferably of approximately 150 to approximately 1,000, and M is a suitable silicone end group, as it is known from the prior art, preferably trimethylsiloxy. Non-limiting examples of the groups represented by R include alkyl groups such as methyl, ethyl, propyl, isopropyl, isopropyl, butyl, isobutyl, amyl, isoamyl, hexyl, isohexyl and the like; alkenyl groups such as vinyl, halogen vinyl, alkyl vinyl, allyl, halogen allyl, alkyl allyl; cycloalkyl groups such as cyclobutyl, cyclopentyl, cyclohexyl and the like; phenyl groups, benzyl groups, halogen hydrocarbon groups such as 3-chloropropyl, 4-bromobutyl, 3,3,3-trifluoropropyl, chlorocyclohexyl, bromophenyl, chlorophenyl and the like; and sulfurous groups such as mercaptoethyl, mercaptopropyl, mercaptohexyl, mercaptophenyl and the like; R is preferably an alkyl group containing 1 to approximately 6 carbon atoms, and most preferably R is methyl. Examples of R¹ include methylene, ethylene, propylene, hexamethylene, decamethylene, —CH₂CH(CH₃)CH₂—, phenylene, naphthylene, —CH₂CH₂SCH₂CH₂—, —CH₂CH₂OCH₂—, —OCH₂CH₂—, —OCH₂CH₂CH₂—, —CH₂CH(CH₃)C(O)OCH₂—, —(CH₂)₃CC(O)OCH₂CH₂—, —C₆H₄C₆H₄—, —C₆H₄CH₂C₆H₄—; and —(CH₂)₃C(O)SCH₂CH₂—.

Z is an organic, aminofunctional group, comprising at least one functional amino group. One possible formula for Z is NH(CH₂)_{z}NH₂, where z is 1 or greater. Another possible formula for Z is —NH(CH₂)_{z}(CH₂)_{zz}NH, where both z and zz are 1 or greater, independently of one another, wherein this structure comprises diamino ring structures, such as piperazinyl. Z most preferably is a —NHCH₂CH₂NH₂ group. Another possible formula for Z is —N(CH₂)_{z}(CH₂)_{zz}NX₂ or —NX₂, where each X is selected independently from X₂ from the group consisting of hydrogen and alkyl groups having 1 to 12 carbon atoms, and zz is 0.

Q is most preferably a polar, aminofunctional group of formula —CH₂CH₂CH₂NHCH₂CH₂NH₂. In the formulas, “a” takes on values in the range of approximately 0 to approximately 2, “b” takes on values in the range of approximately 2 to approximately 3, “a”+“b” is smaller than or equal to 3, and “c” is a number in the range of approximately 1 to approximately 3. The molar ratio of the RₐQ_{b} SiO_{(4-a-b)/2} units to the R_{c}SiO_{(4-c))/2} units is in the range of approximately 1:2 to 1:65, especially of approximately 1:5 to approximately 1:65, and most preferably of approximately 1:15 to approximately 1:20. If one or more silicones of the above formula are used, the different variable substituents in the above formula may differ from each other in the different silicone components present in the silicone mixture.

Preferred agents contemplated herein are characterized by comprising an aminofunctional silicone of formula (Si-II)

R′ₐG₃₋ₐ-Si(OSiG₂)ₙ-(OSiG_{b}R′_{2-b})ₘ—O-SiG₃₋ₐ-R′ₐ (Si-II),

in which the following meanings apply:
G is —H, a phenyl group, —OH, —O—CH₃, —CH₃, —O—CH₂CH₃, —CH₂CH₃, —O—CH₂CH₂CH₃, —CH₂CH₂CH₃, —O—CH(CH₃)₂, —CH(CH₃)₂, —O—CH₂CH₂CH₂CH₃, —CH₂CH₂CH₂CH₃, —O—CH₂CH(CH₃)₂, —CH₂CH(CH₃)₂, —O—CH(CH₃)CH₂CH₃, —CH(CH₃)CH₂CH₃, —O—C(CH₃)₃, —C(CH₃)₃;
a denotes a number between 0 and 3, in particular 0;
b denotes a number between 0 and 1, in particular 1;
m and n are numbers of which the sum (m+n) is between 1 and 2000, especially between 50 and 150, wherein n preferably takes on values from 0 to 1999, and in particular from 49 to 149, and m especially takes on values from 1 to 2000, and in particular from 1 to 10;
R′ is a monovalent group selected from:
   -Q-N(R″)—CH₂—CH₂—N(R″)₂
   -Q-N(R″)₂
   -Q-N⁺(R″)₃A⁻
   -Q-N⁺H(R″)₂
   -Q-N⁺H₂(R″)A⁻
   -Q-N(R″)—CH₂—CH₂—N⁺R″H₂A⁻,
wherein every Q denotes a chemical bond, —CH₂—, —CH₂—CH₂—, —CH₂CH₂CH₂—, —CH₂CH₂CH₂CH₂—, —CH₂C(CH₃)₂—, —CH(CH₃)CH₂CH₂—,
R″ denotes identical or different groups from the group consisting of —H, -phenyl, -benzyl, —CH₂—CH(CH₃)Ph, the C₁₋₂₀ alkyl groups, preferably —CH₃, —CH₂CH₃, —CH₂CH₂CH₃, —CH(CH₃)₂, —CH₂CH₂CH₂H₃, —CH₂CH(CH₃)₂, —CH(CH₃)CH₂CH₃, —C(CH₃)₃, and A represents an anion, which is preferably selected from chloride, bromide, iodide or methosulfate.

Particularly preferred agents contemplated herein are characterized by comprising at least one aminofunctional silicone of formula (Si-IIa)

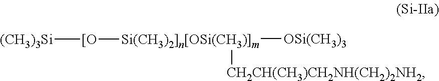

where m and n are numbers of which the sum (m+n) is between 1 and 2000, especially between 50 and 150, wherein n preferably takes on values from 0 to 1999, and in particular from 49 to 149, and m especially takes on values from 1 to 2000, and in particular from 1 to 10.

According to INCI nomenclature, these silicones are referred to as Trimethylsilylamodimethicone.

Particularly preferred are also agents as contemplated herein that comprise an aminofunctional silicone of formula (Si-IIb)

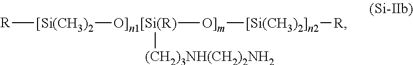

where R denotes —OH, —O—CH₃ or a —CH₃ group, and m, n1 and n2 are numbers of which the sum (m+n1+n2) is between 1 and 2000, especially between 50 and 150, wherein the sum (n1+n2) preferably takes on values from 0 to 1999, and in particular from 49 to 149, and m especially takes on values from 1 to 2000, and in particular from 1 to 10.

According to INCI nomenclature, these silicones are referred to as Amodimethicone.

Regardless of which aminofunctional silicones are used, agents contemplated herein that comprise an aminofunctional silicone having an amine value above about 0.25 meq/g, especially above about 0.3 meq/g, and in particular above about 0.4 meq/g, are preferred. The amine value denotes the milliequivalents of amine per gram of the aminofunctional silicone. This value can be determined by titration and also be described by the unit mg KOH/g.

Preferred agents as contemplated herein are characterized by comprising, based on the weight thereof, about 0.01 to about 20 wt. %, especially about 0.1 to about 10 wt. %, particularly preferably about 0.5 to about 7.5 wt. %, and in particular about 1 to about 5 wt. % aminofunctional silicone(s).

It is particularly preferred if the agents contemplated herein comprise aminofunctional silicone(s) including a terminal hydroxy group or terminal hydroxy groups. Several special aminofunctional silicone(s) including a terminal hydroxy group or terminal hydroxy groups have found to be particularly suitable in the agents as contemplated herein. These are described hereafter.

Preferred agents as contemplated herein are characterized by comprising, based on the weight thereof, about 0.01 to about 20 wt. %, especially about 0.1 to about 10 wt. %, particularly preferably about 0.5 to about 7.5 wt. %, and in particular about 1 to about 5 wt. % of at least one silicone of the following formula (Si-III)

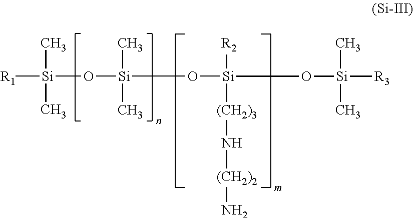

in which
m and n denote numbers selected so that the sum (n+m) is in the range of 1 to 1000;
n is a number in the range of 0 to 999, and m is a number in the range of 1 to 1000;
R1, R2 and R3, which are the same or different, denote a hydroxy group or a C1-4 alkoxy group;
wherein at least one of the groups R1 to R3 denotes a hydroxy group.

Further preferred agents contemplated herein are characterized by comprising, based on the weight thereof, about 0.01 to about 20 wt. %, especially about 0.1 to about 10 wt. %, particularly preferably about 0.5 to about 7.5 wt. %, and in particular about 1 to about 5 wt. % of at least one silicone of the following formula (Si-IV)

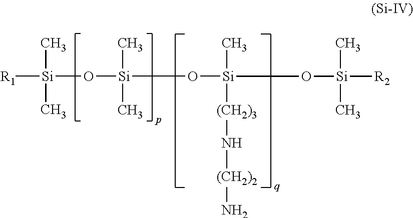

in which
p and q denote numbers selected so that the sum (p+q) is in the range of 1 to 1000;
p is a number in the range of 0 to 999, and q is a number in the range of 1 to 1000;
R1 and R2, which are different from each other, denote a hydroxy group or a C1-4 alkoxy group, wherein at least one of the groups R1 to R2 denotes a hydroxy group.

The silicones of formulas (Si-Ill) and (Si-IV) differ from each other by the grouping at the Si atom, which carries the nitrogen-containing group. In formula (Si-Ill), R2 denotes a hydroxy group or a C1-4 alkoxy group, while the group is a methyl group in formula (Si-IV). The individual Si groupings, which are identified by the subscripts m and n, or p and q, do not have to be present in the form of blocks; rather, the individual units may also be statistically distributed, which is to say not necessarily every R1-Si(CH₃)₂ group is bound to a —[O—Si(CH₃)₂]— grouping in formulas (Si-III) and (Si-IV).

In the method as contemplated herein, pretreatment agents that have proven to be particularly effective with respect to the desired outcome are those that comprise at least one silicone of formula (Si-V):

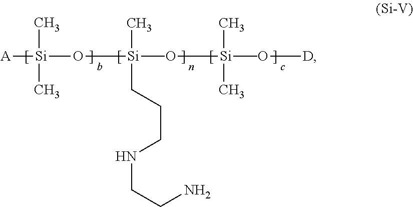

in which
A denotes a group —OH, —O—Si(CH₃)₃, —O—Si(CH₃)₂OH, —O—Si(CH₃)₂OCH₃;
D denotes a group —H, —Si(CH₃)₃, —Si(CH₃)₂OH, —Si(CH₃)₂OCH₃;
b, n and c denote integers between 0 and 1000;
   with the proviso that
      n>0 and b+c>0
      at least one of the conditions A=—OH and D=—H is met.

Agents as contemplated herein that, based on the weight thereof, comprise about 0.01 to about 20 wt. %, especially about 0.1 to about 10 wt. %, particularly preferably about 0.5 to about 7.5 wt. %, and in particular about 1 to about 5 wt. % of at least one silicone of formula (Si-V):

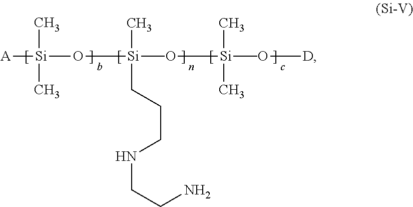

in which
A denotes a group —OH, —O—Si(CH₃)₃, —O—Si(CH₃)₂OH, —O—Si(CH₃)₂OCH₃;
D denotes a group —H, —Si(CH₃)₃, —Si(CH₃)₂OH, —Si(CH₃)₂OCH₃;
b, n and c denote integers between 0 and 1000;
   with the proviso that
      n>0 and b+c>0
      at least one of the conditions A=—OH and D=—H is met,
         are thus preferred.

In the above-described formula (Si-V), the individual siloxane units having the subscripts b, c and n are statistically distributed, which is to say these do not necessarily have to be block copolymers.

Further particularly suitable silicones are 4-morpholinomethyl-substituted. Agents contemplated herein that, based on the weight thereof, comprise about 0.01 to about 20 wt. %, especially about 0.1 to about 10 wt. %, particularly preferably about 0.5 to about 7.5 wt. %, and in particular about 1 to about 5 wt. % of at least one 4-morpholinomethyl-substituted silicone of formula (Si-V):

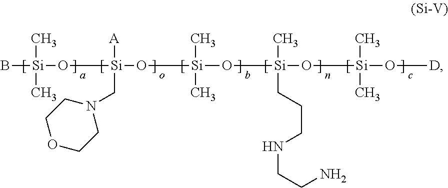

in which
A denotes a structural unit (i) bound via an —O—

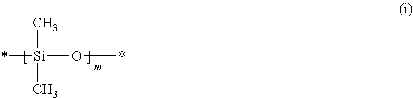

or an oligomeric or polymeric group bound via an —O— and containing structural units of formula (i), or —OH;
denotes a bond to the structural unit (i), or a terminal group B (Si-bound) or D (O-bound);
B denotes a group —OH, —O—Si(CH₃)₃, —O—Si(CH₃)₂OH, —O—Si(CH₃)₂OCH₃;
D denotes a group —H, —Si(CH₃)₃, —Si(CH₃)₂OH, —Si(CH₃)₂OCH₃;
a, b and c denote integers between 0 and 1000, with the proviso that a+b+c>0;
m, n and o denote integers between 1 and 1000,
with the proviso that at least one of the conditions B=—OH and D=—H is met,
   are particularly preferred.

Chemical formula (Si-VI) is intended to illustrate that the siloxane groups n and o do not necessarily have to be bound directly to a terminal group B or D. Rather, in preferred formulas (Si-VI) a>0 or b>0 applies, and in particularly preferred formulas (Si-VI) a>0 and b>0 applies, which is to say the terminal group B or D is preferably bound to a dimethylsiloxy group. In formula (Si-VI), the siloxane units a, b, c, n and o are also preferably statistically distributed. The silicones represented by formula (Si-VI) and used as contemplated herein may be trimethylsilyl-terminated (D or B=—Si(CH₃)₃); however, they may also be dimethylsilylhydroxy-terminated on both sides, or dimethylsilylhydroxy- and dimethylsilylmethoxy-terminated on one side. Silicones used particularly preferably as contemplated herein are selected from silicones in which the following meanings apply:
B=—O—Si(CH₃)₂OH and D=—Si(CH₃)₃
B=—O—Si(CH₃)₂OH and D=—Si(CH₃)₂OH
B=—O—Si(CH₃)₂OH and D=—Si(CH₃)₂OCH₃
B=—O—Si(CH₃)₃ and D=—Si(CH₃)₂OH
B=—O—Si(CH₃)₂OCH₃ and D=—Si(CH₃)₂OH.
   These silicones result in exorbitant improvements in the properties of hair treated with the agents contemplated herein, in particular in in significantly improved protection during oxidative treatment. In formula (Si-VI), group A may denote
a structural unit (i) bound via an —O— or
an oligomeric or polymeric group bound via an —O— and containing structural units of formula (i),
or —OH.

Formula (Si-VI) is thus defined more precisely as one of formulas (Si-VIa), (Si-VIb) or (Si-VIc):

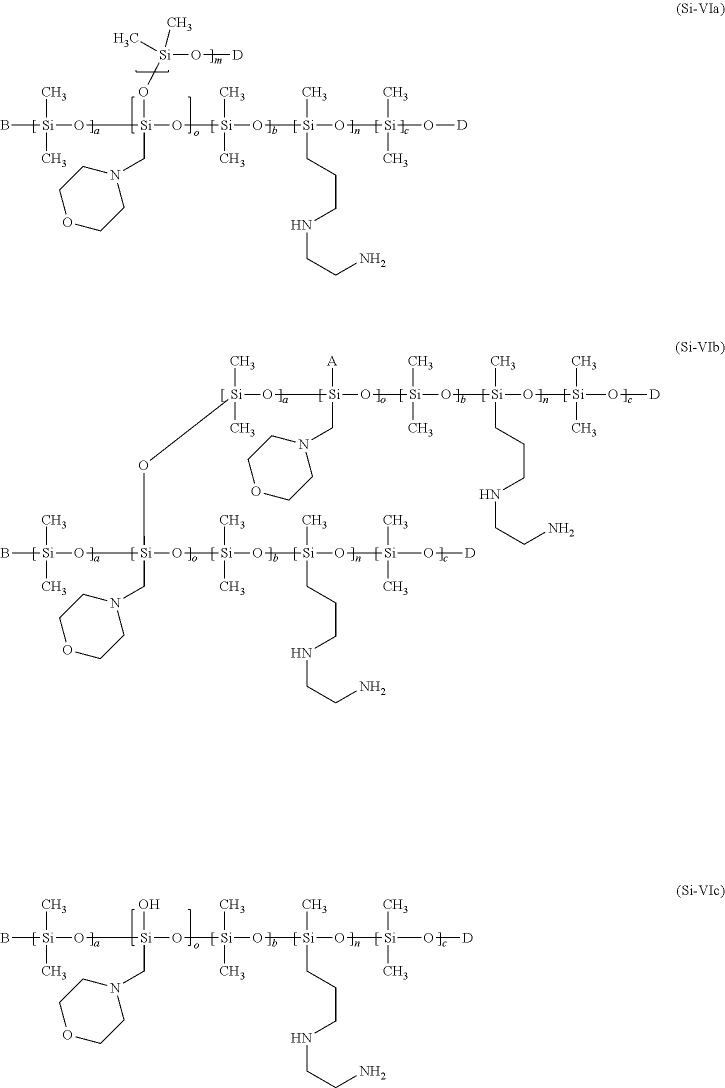

Regardless of what type of the aminofunctional silicone(s) having a terminal hydroxy group or terminal hydroxy groups is used, the agents contemplated herein preferably comprise the silicone(s) in the form of an emulsion, and particularly preferably in the form of a microemulsion.

It has been shown that the action of the silicones used in the agents contemplated herein can be further increased when certain non-ionic components are likewise used in the agents. Moreover, these non-ionic components have positive effects on the shelf life of the agents. Non-ionic components that are particularly suited here are ethoxylates of decanol, undecanol, dodecanol, tridecanol and so forth. Ethoxylated tridecanols have proven to be particularly suited, which particularly preferably are incorporated into the agents contemplated herein. Particularly preferred agents contemplated herein are characterized by comprising, based on the weight thereof, about 0.00001 to about 5 wt. %, especially about 0.0001 to about 3.5 wt. %, particularly preferably about 0.001 to about 2 wt. %, more preferably about 0.01 to about 1 wt. %, and in particular about 0.1 to about 0.5 wt. % branched, ethoxylated tridecanol (INCI name: Trideceth-5) or α-iso-tridecyl-w-hydroxy polyglycol ether (INCI name: Trideceth-10) or the mixtures thereof.

The hair treatment agents contemplated herein can comprise vegetable oils, vegetable butters and/or vegetable waxes. These vegetable oil components impart improved combing and styling properties to the hair and increased shine.

Suitable vegetable oil components include natural (vegetable) oils and/or butters, which typically comprise triglycerides and mixtures of triglycerides. Preferred natural oils are coconut oil, (sweet) almond oil, walnut oil, peach kernel oil, apricot kernel oil, argan oil, avocado oil, tea tree oil, soy bean oil, sesame oil, sunflower oil, tsubaki oil, evening primrose oil, rice bran oil, palm kernel oil, mango kernel oil, marula oil, lady's smock oil, thistle oil, macadamia nut oil, grape seed oil, amaranth seed oil, bamboo oil, olive oil, wheat germ oil, pumpkin seed oil, mallow oil, hazelnut oil, safflower oil, canola oil, sasanqua oil, jojoba oil, rambutan oil, cocoa butter and/or shea butter.

Suitable natural or vegetable waxes that can preferably be used are carnauba waxes, beeswaxes and/or candelilla waxes.

Particularly preferred vegetable oil components are (sweet) almond oil, peach kernel oil, apricot kernel oil, amaranth seed oil, argan oil, olive oil, jojoba oil, cocoa butter and/or shea butter. In particular, apricot kernel oil, argan oil, olive oil and/or jojoba oil are preferred.

In a preferred embodiment, the hair treatment agents contemplated herein preferably comprise coconut oil, (sweet) almond oil, walnut oil, peach kernel oil, apricot kernel oil, argan oil, avocado oil, tea tree oil, soy bean oil, sesame oil, sunflower oil, tsubaki oil, evening primrose oil, rice bran oil, palm kernel oil, mango kernel oil, marula oil, lady's smock oil, thistle oil, macadamia nut oil, grape seed oil, amaranth seed oil, bamboo oil, olive oil, wheat germ oil, pumpkin seed oil, mallow oil, hazelnut oil, safflower oil, canola oil, sasanqua oil, jojoba oil, rambutan oil, cocoa butter and/or shea butter.

Within this embodiment, it is particularly preferred if the hair treatment agents contemplated herein comprise (sweet) almond oil, peach kernel oil, apricot kernel oil, amaranth seed oil, argan oil, olive oil, jojoba oil, cocoa butter and/or shea butter.

The percentage of weight of the at least one vegetable oil, vegetable butter and/or vegetable wax based on the total weight of the hair treatment agent contemplated herein is preferably about 0.02 to about 2.50 wt. %, more preferably about 0.03 to about 2.00 wt. %, particularly preferably about 0.04 to about 1.50 wt. %, and in particular about 0.05 to about 1.00 wt. %.

In addition to the above-mentioned essential and optional components, in one further preferred embodiment the hair treatment agents as contemplated herein may include at least one further hair conditioning active ingredient so as to further enhance the nourishing properties of the agents, which may be selected from the group consisting of:
protein hydrolysates;
vitamins;
plant extracts and/or
glycerol.
   Suitable protein hydrolysates shall be understood to mean product mixtures that can be obtained by the acidically, basically or enzymatically catalyzed degradation of proteins. Protein hydrolysates of plant, animal and/or marine origin can be used. Animal protein hydrolysates are, for example, elastin, collagen, keratin, silk and milk protein hydrolysates, which can also be present in the form of salts. Such products are sold, for example, under the trademarks Dehylan® (Cognis), Promois® (Interorgana), Collapuron® (Cognis), Nutrilan® (Cognis), Gelita-Sol® (Deutsche Gelatine Fabriken Stoess & Co), Lexein® (Inolex), and Kerasol® (Croda). Protein hydrolysates of plant origin, such as soy, almond, rice, pea, potato and wheat protein hydrolysates, are preferred. Such products are available, for example, under the trademarks Gluadin® (Cognis), DiaMin® (Diamalt), Lexein® (Inolex), and Crotein® (Croda). It is also possible to use cationized protein hydrolysates, wherein the underlying protein hydrolysate can stem from animals, for example from collagen, milk or keratin, from plants, for example from wheat, maize, rice, potatoes, soy or almonds, from marine life forms, for example from fish collagen or algae, or from protein hydrolysates obtained by way of biotechnology. The protein hydrolysates underlying the cationic derivatives can be obtained from the corresponding proteins by way of chemical, in particular alkaline or acid hydrolysis, enzymatic hydrolysis and/or a combination of the two types of hydrolysis. The hydrolysis of proteins generally results in a protein hydrolysate having a molecular weight distribution from approximately 100 daltons to several thousand daltons Cationic protein hydrolysates in which the underlying protein component has a molecular weight of about 100 to about 25,000 daltons, and preferably about 250 to about 5000 daltons, are preferred. Moreover, cationic protein hydrolysates shall be understood to mean quaternized amino acids and the mixtures thereof. The quaternization of the protein hydrolysates or of the amino acids is frequently carried out using quaternary ammonium salts, such as N,N-dimethyl-N-(n-alkyl)-N-(2-hydroxy-3-chloro-n-propyl) ammonium halides. The cationic protein hydrolysates can moreover also be further derivatized. Typical examples of the cationic protein hydrolysates and derivatives that shall be mentioned are the commercially available products known under the following INCI names: Cocodimonium Hydroxypropyl Hydrolyzed Collagen, Cocodimonium Hydroxypropyl Hydrolyzed Casein, Cocodimonium Hydroxypropyl Hydrolyzed Collagen, Cocodimonium Hydroxypropyl Hydrolyzed Hair Keratin, Cocodimonium Hydroxypropyl Hydrolyzed Keratin, Cocodimonium Hydroxypropyl Hydrolyzed Rice Protein, Cocodimonium Hydroxypropyl Hydrolyzed Silk, Cocodimonium Hydroxypropyl Hydrolyzed Soy Protein, Cocodimonium Hydroxypropyl Hydrolyzed Wheat Protein, Cocodimonium Hydroxypropyl Silk Amino Acids, Hydroxypropyl Arginine Lauryl/Myristyl Ether HCl, Hydroxypropyltrimonium Gelatin, Hydroxypropyltrimonium Hydrolyzed Casein, Hydroxypropyltrimonium Hydrolyzed Collagen, Hydroxypropyltrimonium Hydrolyzed Conchiolin Protein, Hydroxypropyltrimonium Hydrolyzed keratin, Hydroxypropyltrimonium Hydrolyzed Rice Bran Protein, Hydroxypropyltrimonium Hydrolyzed Silk, Hydroxypropyltrimonium Hydrolyzed Soy Protein, Hydroxypropyl Hydrolyzed Vegetable Protein, Hydroxypropyltrimonium Hydrolyzed Wheat Protein, Hydroxypropyltrimonium Hydrolyzed Wheat Protein/Siloxysilicate, Laurdimonium Hydroxypropyl Hydrolyzed Soy Protein, Laurdimonium Hydroxypropyl Hydrolyzed Wheat Protein, Laurdimonium Hydroxypropyl Hydrolyzed Wheat Protein/Siloxysilicate, Lauryldimonium Hydroxypropyl Hydrolyzed Casein, Lauryldimonium Hydroxypropyl Hydrolyzed Collagen, Lauryldimonium Hydroxypropyl Hydrolyzed Keratin, Lauryldimonium Hydroxypropyl Hydrolyzed Silk, Lauryldimonium Hydroxypropyl Hydrolyzed Soy Protein, Steardimonium Hydroxypropyl Hydrolyzed Casein, Steardimonium Hydroxypropyl Hydrolyzed Collagen, Steardimonium Hydroxypropyl Hydrolyzed Keratin, Steardimonium Hydroxypropyl Hydrolyzed Rice Protein, Steardimonium Hydroxypropyl Hydrolyzed Silk, Steardimonium Hydroxypropyl Hydrolyzed Soy Protein, Steardimonium Hydroxypropyl Hydrolyzed Vegetable Protein, Steardimonium Hydroxypropyl Hydrolyzed Wheat Protein, Steartrimonium Hydroxyethyl Hydrolyzed Collagen, Quaternium-76 Hydrolyzed Collagen, Quaternium-79 Hydrolyzed Collagen, Quaternium-79 Hydrolyzed Keratin, Quaternium-79 Hydrolyzed Milk Protein, Quaternium-79 Hydrolyzed Silk, Quaternium-79 Hydrolyzed Soy Protein, Quaternium-79 Hydrolyzed Wheat Protein.

The percentage by weight of the protein hydrolysate or protein hydrolysates based the total weight of the hair treatment agents is preferably about 0.01 to about 5 wt. %, more preferably about 0.025 to about 3 wt. %, and in particular about 0.05 to about 2 wt. %.

Regardless of the source (vegetable, animal, maritime and the like), protein hydrolysates comprise individual amino acids, oligopeptides and optionally polypeptides, depending on the degree of hydrolysis.

It is particularly preferred if the hair treatment agents contemplated herein comprise at least one oligopeptide including at least one amino acid sequence Glu-Glu-Glu

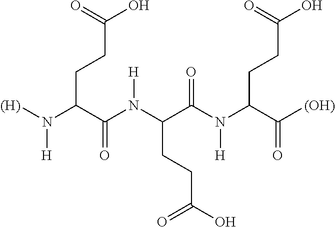

wherein the amino group may be present in a free or protonated state, and the carboxy groups may be present in a free or deprotonated state.

Preferred hair treatment agents according to the invention are characterized by comprising, based on the weight thereof, about 0.0001 to about 10 wt. % of at least one oligopeptide including at least one amino acid sequence Glu-Glu-Glu

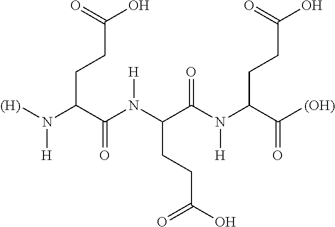

wherein the amino group may be present in a free or protonated state, and the carboxy groups may be present in a free or deprotonated state.

In this formula, as in all formulas hereafter, the bracketed hydrogen atom of the amino group as well as the bracketed hydroxy group of the acid function mean that the groups in question may be present per se (this involves an oligopeptide having the relevant number of amino acids, as shown) (3 in the formula above), or else that the amino acid sequence is present in an oligopeptide that includes further amino acids; depending on where the further amino acid or amino acids is or are bound, the bracketed components of the above-mentioned formula are replaced by the further amino acid group(s).

These preferred hair treatment agents as contemplated herein comprise, based on the weight thereof, about 0.0001 to about 10 wt. %, of at least one oligopeptide including at least one amino acid sequence Glu-Glu-Glu, which is to say at least three consecutive glutamic acids.

Oligopeptides within the meaning of the present application are condensation products of amino acids comprising at least 3 and no more than 25 amino acids, the condensation products being linked by peptide bonds in an acid amide-like manner. In preferred hair treatment agents contemplated herein, the oligopeptide comprises 5 to 15 amino acids, especially 6 to 13 amino acids, particularly preferably 7 to 12 amino acids, and in particular 8, 9 or 10 amino acids.

Depending on whether further amino acids are bound to the Glu-Glu-Glu sequence, and depending on the type of these amino acids, the molar mass of the oligopeptide present in the agents contemplated herein may vary. Preferred hair treatment agents used as contemplated herein are characterized in that the oligopeptide has a molar mass of about 650 to about 3000 Da, especially of about 750 to about 2500 Da, particularly preferably of about 850 to about 2000 Da, and in particular of about 1000 to about 1600 Da.

In summary, preferred hair treatment agents are characterized in that the oligopeptide comprises 5 to 15 amino acids, especially 6 to 13 amino acids, particularly preferably 7 to 12 amino acids, and in particular 8, 9 or 10 amino acids, and has a molar mass of about 650 to about 3000 Da, especially of about 750 to about 2500 Da, particularly preferably of about 850 to about 2000 Da, and in particular of about 1000 to about 1600 Da.

As is apparent from the preferred number of amino acids in the oligopeptides and the preferred molar mass range, oligopeptides that are not composed solely of the three glutamic acids, but comprise further amino acids bound to this sequence are preferred. These further amino acids are preferably selected from certain amino acids, while certain other representatives are less preferred.

It is preferred, for example, if the oligopeptides used in the agents contemplated herein do not contain any methionine.

It is further preferred if the oligopeptides used in the agents contemplated herein do not contain any cysteine and/or cystine.

It is further preferred if the oligopeptides used in the agents contemplated herein do not contain any aspartic acid and/or asparagine.

It is further preferred if the oligopeptides used in the agents contemplated herein do not contain any serine and/or threonine.

In contrast, it is preferred if the oligopeptides used in the agents contemplated herein contain tyrosine.

It is further preferred if the oligopeptides used in the agents contemplated herein contain leucine.

It is further preferred if the oligopeptides used in the agents contemplated herein contain isoleucine.

It is further preferred if the oligopeptides used in the agents contemplated herein contain arginine.

It is further preferred if the oligopeptides used in the agents contemplated herein contain valine.

Particularly preferred oligopeptides, or amino acid sequences present in the preferred oligopeptides, are described hereafter.

A particularly preferred oligopeptide additionally comprises tyrosine, which is preferably bound to the Glu-Glu-Glu sequence via the acid function. Preferred hair treatment agents as contemplated herein are thus characterized in that the oligopeptide present therein includes at least one amino acid sequence Tyr-Glu-Glu-Glu

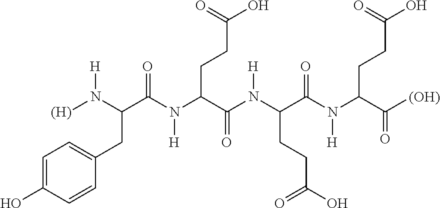

wherein the amino group may be present in a free or protonated state, and the carboxy groups may be present in a free or deprotonated state.

A further particularly preferred oligopeptide additionally comprises isoleucine, which is preferably bound to the Glu-Glu-Glu sequence via the amino function. Preferred hair treatment agents contemplated herein are thus characterized in that the oligopeptide present therein includes at least one amino acid sequence Glu-Glu-Glu-Ile

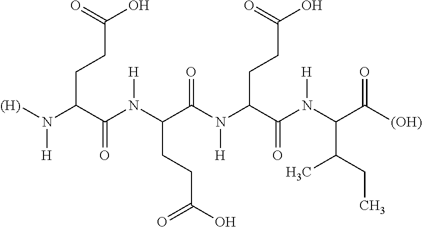

wherein the amino group may be present in a free or protonated state, and the carboxy groups may be present in a free or deprotonated state.

Oligopeptides that include both of the above-mentioned amino acids (tyrosine and isoleucine) are preferred. Particularly preferred are hair treatment agents contemplated herein in which the oligopeptide present in the hair treatment agent includes at least one amino acid sequence Tyr-Glu-Glu-Glu-Ile

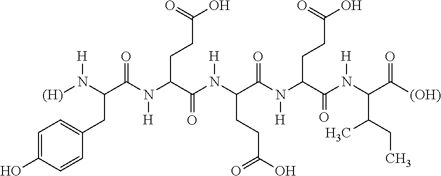

wherein the amino group may be present in a free or protonated state, and the carboxy groups may be present in a free or deprotonated state.

Further preferred oligopeptides additionally comprise arginine, which is preferably present bound to isoleucine.

Particularly preferred are hair treatment agents contemplated herein in which the oligopeptide present in the hair treatment agent includes at least one amino acid sequence Tyr-Glu-Glu-Glu-Ile-Arg

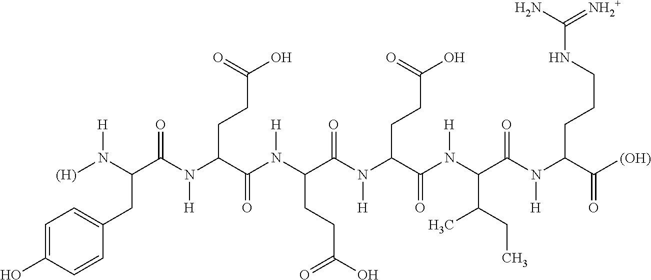

wherein the amino groups may be present in a free or protonated state, and the carboxy groups may be present in a free or deprotonated state.

Still further preferred oligopeptides additionally comprise valine, which is preferably present bound to the arginine. Further preferred hair treatment agents contemplated herein are thus characterized in that the oligopeptide present in the hair treatment agent includes at least one amino acid sequence Tyr-Glu-Glu-Glu-Ile-Arg-Val

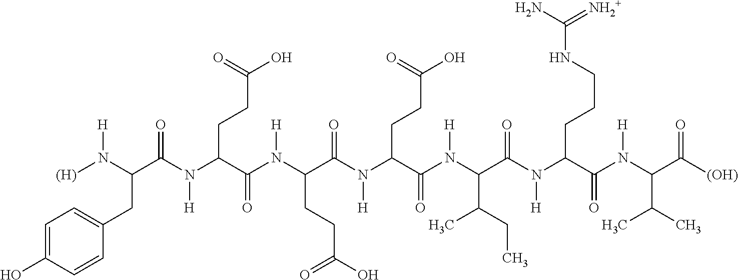

wherein the amino groups may be present in a free or protonated state, and the carboxy groups may be present in a free or deprotonated state.

Still further preferred oligopeptides additionally comprise leucine, which is preferably present bound to the valine. Further preferred hair treatment agents according to the invention are thus characterized in that the oligopeptide present in the hair treatment agent includes at least one amino acid sequence Tyr-Glu-Glu-Glu-Ile-Arg-Val-Leu

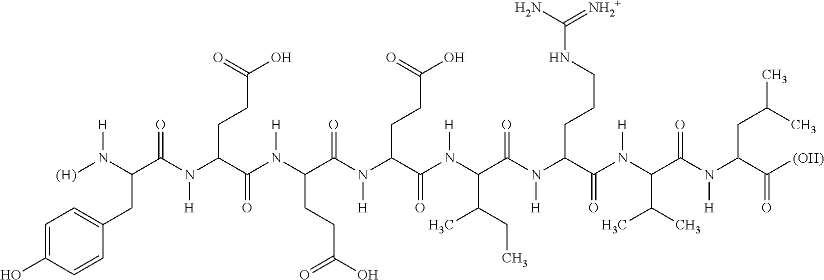

wherein the amino groups may be present in a free or protonated state, and the carboxy groups may be present in a free or deprotonated state.

In particular, preferred oligopeptides additionally comprise leucine, which is preferably present bound to the tyrosine. Further preferred hair treatment agents contemplated herein are thus characterized in that the oligopeptide present in the hair treatment agent includes at least one amino acid sequence Leu-Tyr-Glu-Glu-Glu-Ile-Arg-Val-Leu

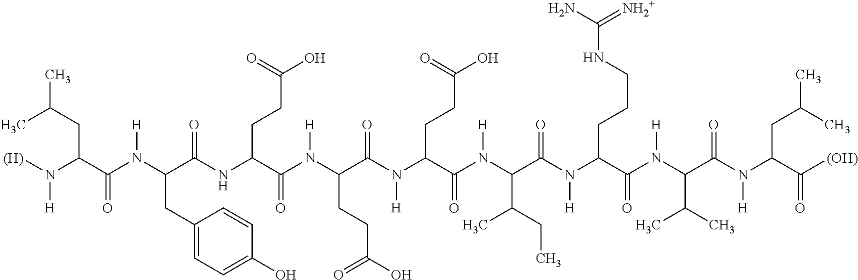

wherein the amino groups may be present in a free or protonated state, and the carboxy groups may be present in a free or deprotonated state.

It is especially particularly preferred if agents contemplated herein comprise at least two oligopeptides that satisfy the above-described criteria, but differ from one another. For example, the use of hair treatment agents comprising at least two oligopeptides A and B that are different from one another, but both include the amino acid sequence Glu-Glu-Glu, is preferred.

These oligopeptides A and B that are different from one another correspond to each other in that these carry three consecutive Glu amino acids in the amino acid sequence, while they have differences in the amino acids bound before or after that. Preferred are peptides that are different from one another and exhibit partial agreement with each other, which may also be greater than the above-mentioned three amino acids.

For example, further preferred hair treatment agents are characterized in that at least two oligopeptides A and B that are different from one another, but both include the amino acid sequence Glu-Glu-Glu-Ile, are present in the hair treatment agent.

Hair treatment agents comprising at least two oligopeptides A and B that are different from one another, but both include the amino acid sequence Ty-Glu-Glu-Glu, are likewise preferred.

Still further preferred hair treatment agents are characterized by comprising at least two oligopeptides A and B that are different from one another, but both include the amino acid sequence Glu-Glu-Glu-Ile-Arg.

Likewise, still further preferred hair treatment agents are characterized in that at least two oligopeptides A and B that are different from one another, but both include the amino acid sequence Tyr-Glu-Glu-Glu-Ile are present in the hair treatment agent.

Preferred hair treatment agents contemplated herein are thus characterized in that the oligopeptide includes at least one amino acid sequence Tyr-Glu-Glu-Glu-Ile

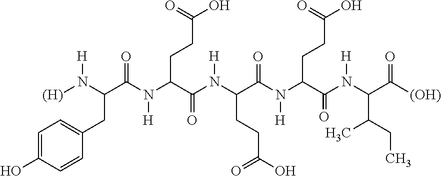

wherein the amino group may be present in a free or protonated state, and the carboxy groups may be present in a free or deprotonated state.

Especially particularly preferred hair treatment agents are characterized in that at least two oligopeptides A and B that are different from one another, but both include the amino acid sequence Glu-Glu-Glu-Ile-Arg are present in the hair treatment agent

Likewise, especially particularly preferred hair treatment agents are characterized in that at least two oligopeptides A and B that are different from one another, but both include the amino acid sequence Tyr-Glu-Glu-Glu-Ile-Arg are present in the hair treatment agent

The oligopeptides preferably have an even greater structural agreement. For example, hair treatment agents comprising at least two oligopeptides A and B that are different from one another, but both include the amino acid sequence Glu-Glu-Glu-Ile-Arg-Val, are further preferred embodiments.

Hair treatment agents comprising at least two oligopeptides A and B that are different from one another, but both include the amino acid sequence Tyr-Glu-Glu-Glu-Ile-Arg-Val, are likewise preferred embodiments.

Still further preferred hair treatment agents contemplated herein are characterized by comprising at least two oligopeptides A and B that are different from one another, but both include the amino acid sequence Glu-Glu-Glu-Ile-Arg-Val-Leu.

Likewise, still further preferred hair treatment agents contemplated herein are characterized by comprising at least two oligopeptides A and B that are different from one another, but both include the amino acid sequence Tyr-Glu-Glu-Glu-Ile-Arg-Val-Leu.

Preferred hair treatment agents as contemplated herein are thus characterized in that the oligopeptide includes at least one amino acid sequence Tyr-Glu-Glu-Glu-Ile-Arg-Val-Leu

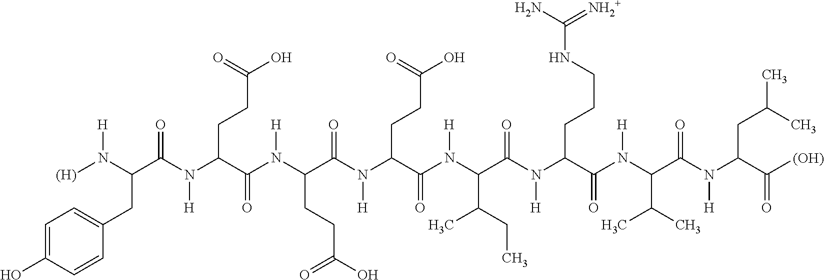

wherein the amino groups may be present in a free or protonated state, and the carboxy groups may be present in a free or deprotonated state.

In particular, preferred hair treatment agents contemplated herein are characterized by comprising at least two oligopeptides A and B that are different from one another, wherein oligopeptide A includes the amino acid sequence Leu-Tyr-Glu-Glu-Glu-Ile-Arg-Val-Leu

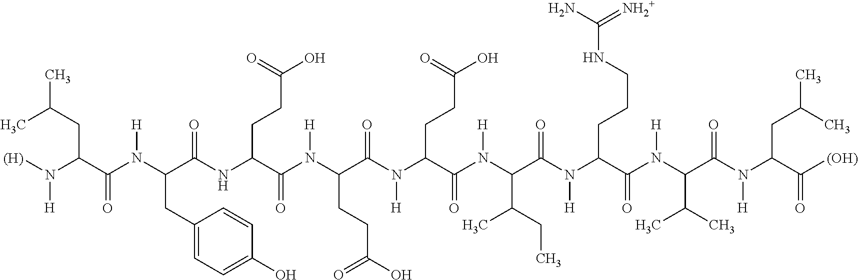

wherein the amino groups may be present in a free or protonated state, and the carboxy groups may be present in a free or deprotonated state,
and oligopeptide B includes the amino acid sequence Tyr-Glu-Glu-Glu-Ile-Arg-Val-Leu

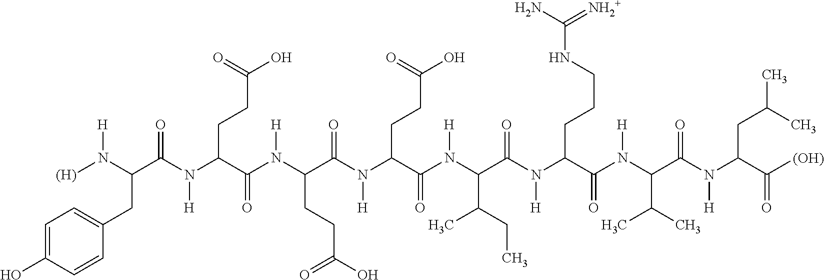

wherein the amino groups may be present in a free or protonated state, and the carboxy groups may be present in a free or deprotonated state.

In especially particularly preferred hair treatment agents of this last embodiment, these comprise, based on the weight of the agent, about 0.00001 to about 1 wt. % oligopeptide A and about 0.00001 to about 1 wt. % oligopeptide B.

In further preferred hair treatment agents of this last embodiment, these comprise, based on the weight of the agent, about 0.00005 to 0. about 1 wt. % oligopeptide A and about 0.00005 to about 0.1 wt. % oligopeptide B.

In still further preferred hair treatment agents of this last embodiment, these comprise, based on the weight of the agent, about 0.0001 to about 0.01 wt. % oligopeptide A and about 0.0001 to about 0.001 wt. % oligopeptide B.

The oligopeptides that are used herein and satisfy the above-described conditions can advantageously be obtained from keratinic materials. It is preferred that these oligopeptides are used in high proportions, based on the entire keratinic peptide content of the agents.

It is especially particularly preferred that a preferably high proportion of all keratinic peptides present in the agent as contemplated herein satisfies the above-described conditions.

Preferred hair treatment agents as contemplated herein are characterized in that at least about 0.1 wt. %, especially at least about 0.5 wt. %, particularly preferably at least about 1 wt. %, more preferably at least about 2.5 wt. %, still more preferably at least about 5 wt. %, and in particular at least about 10 wt. % of all keratinic peptides present in the agent include the amino acid sequence Glu-Glu-Glu.

Further preferred hair treatment agents as contemplated herein are characterized in that at least about 0.1 wt. %, especially at least about 0.5 wt. %, particularly preferably at least about 1 wt. %, more preferably at least about 2.5 wt. %, still more preferably at least about 5 wt. %, and in particular at least about 10 wt. % of all keratinic peptides present in the agent include the amino acid sequence Glu-Glu-Glu-Ile.

Still further preferred hair treatment agents as contemplated herein are characterized in that at least about 0.1 wt. %, especially at least about 0.5 wt. %, particularly preferably at least about 1 wt. %, more preferably at least about 2.5 wt. %, still more preferably at least about 5 wt. %, and in particular at least about 10 wt. % of all keratinic peptides present in the agent include the amino acid sequence Tyr-Glu-Glu-Glu.

Particularly preferred hair treatment agents as contemplated herein are characterized in that at least about 0.1 wt. %, especially at least about 0.5 wt. %, particularly preferably at least about 1 wt. %, more preferably at least about 2.5 wt. %, still more preferably at least about 5 wt. %, and in particular at least about 10 wt. % of all keratinic peptides present in the agent include the amino acid sequence Tyr-Glu-Glu-Glu-Ile.

Especially particularly preferred hair treatment agents as contemplated herein are characterized in that at least about 0.1 wt. %, especially at least about 0.5 wt. %, particularly preferably at least about 1 wt. %, more preferably at least about 2.5 wt. %, still more preferably at least about 5 wt. %, and in particular at least about 10 wt. % of all keratinic peptides present in the agent include the amino acid sequence Tyr-Glu-Glu-Glu-Ile-Arg.

Still further preferred hair treatment agents as contemplated herein are characterized in that at least about 0.1 wt. %, especially at least about 0.5 wt. %, particularly preferably at least about 1 wt. %, more preferably at least about 2.5 wt. %, still more preferably at least about 5 wt. %, and in particular at least about 10 wt. % of all keratinic peptides present in the agent include the amino acid sequence Tyr-Glu-Glu-Glu-Ile-Arg-Val.

In particular, preferred hair treatment agents as contemplated herein are characterized in that at least about 0.1 wt. %, especially at least about 0.5 wt. %, particularly preferably at least about 1 wt. %, more preferably at least about 2.5 wt. %, still more preferably at least about 5 wt. %, and in particular at least about 10 wt. % of all keratinic peptides present in the agent include the amino acid sequence Tyr-Glu-Glu-Glu-Ile-Arg-Val-Leu.

The above-described conditions relate to the total content of peptides derived from keratinic materials in the agents as contemplated herein. In addition to the oligopeptides of keratinic origin, it is possible, of course, to use further peptides and/or protein hydrolysates, for example from other native sources. For example, the additional use of wheat protein hydrolysates is preferred.

Suitable vitamins shall preferably be understood to mean the following vitamins, provitamins, and vitamin precursors, and the derivatives thereof:
Vitamin A: The group of substances referred to as vitamin A includes retinol (vitamin A₁) and 3,4-didehydroretinol (vitamin A₂). β-carotene is the provitamin of retinol. Possible vitamin A components are, for example, vitamin A acid and the esters thereof, vitamin A aldehyde, and vitamin A alcohol and the esters thereof, such as palmitate and acetate.
Vitamin B: The vitamin B group or vitamin B complex includes, among other things.
   Vitamin B₁ (thiamine)
   Vitamin B₂ (riboflavin)
   Vitamin B₃. The compounds nicotinic acid and nicotinic acid amide (niacinamide) are often covered by this designation.
   Vitamin B₅ (pantothenic acid and panthenol). Within this group, panthenol is preferably used. Derivatives of panthenol that can be used are in particular the esters and ethers of panthenol, pantolactone and cationically derivatized panthenols. Individual representatives are, for example, panthenol triacetate, panthenol monoethyl ether and the monoacetate thereof, and cationic panthenol derivatives.
   Vitamin B₆ (pyridoxine, pyridoxamine and pyridoxal).
Vitamin C (ascorbic acid): Use in the form of the palmitic acid ester, glucosides or phosphates can be preferred. Use in combination with tocopherols can likewise be preferred.
Vitamin E (tocopherols, in particular α-tocopherol).
Vitamin F: The term “vitamin F” is usually understood to mean essential fatty acids, in particular linoleic acid, linolenic acid and arachidonic acid.
Vitamin H: Vitamin H refers to the compound (3aS,4S,6aR)-2-oxohexahydrothienol[3,4-d]-imidazole-4-valeric acid, which, however, has since become established by the trivial name biotin.
   Vitamins, provitamins and vitamin precursors from the groups A, B, E and H are particularly preferred. In particular, nicotinic acid amide, biotin, pantolactone and/or panthenol are preferred. The percentage by weight of the vitamin(s), vitamin derivative(s) and/or vitamin precursor(s) based the total weight of the hair treatment agents is preferably about 0.001 to about 2 wt. %, particularly preferably about 0.005 to about 1 wt. %, and in particular about 0.01 to about 0.5 wt. %.

Suitable plant extracts shall be understood to mean extracts that can be produced from all parts of a plant. These extracts are usually produced by extracting the entire plant. However, in isolated cases, it may also be preferred to produce the extracts exclusively from flowers and/or leaves of the plant. Especially suited are the extracts of green tea, oak bark, stinging nettle, witch hazel, hops, chamomile, burdock root, horsetail, hawthorn, lime blossom, lychee, almond, aloe vera, pine needle, horse chestnut, sandalwood, juniper, coconut, mango, apricot, lime, wheat, kiwi, melon, orange, grapefruit, sage, rosemary, birch, mallow, lady's smock, creeping thyme, yarrow, thyme, balm, restharrow, coltsfoot, marshmallow, ginseng, ginger root, Echinacea purpurea, Olea europea, Boerhavia diffusa roots, Foeniculum vulgare and Apium graveolens. Particularly preferred for use in the compositions contemplated herein are the extracts of green tea, stinging nettle, witch hazel, chamomile, aloe vera, ginseng, Echinacea purpurea, Olea europea and Boerhavia diffusa roots. Extracting agents for producing the described plant extracts that may be used are water, alcohols and the mixtures thereof. Among the alcohols, lower alcohols, such as ethanol and isopropanol, in particular, however, polyhydric alcohols, such as ethylene glycol and propylene glycol, both as the sole extracting agent and also in a mixture with water, are preferred. Plant extracts based on water/propylene glycol at a ratio of 1:10 to 10:1 have proven to be particularly suitable.

The plant extracts can be used either in pure form or in dilute form. If they are used in dilute form, they usually contain approximately about 2 to 80 wt. % active substance and, as solvent, the extracting agent or extracting agent mixture used during the extraction thereof. The plant extracts may preferably be used in the hair treatment agents contemplated herein (based on the total weight of the agents) in an amount of about 0.01 to about 10 wt. %, more preferably of about 0.05 to about 7.5 wt. %, and in particular of about 0.1 to about 5 wt. %.

Glycerol can be added separately to the hair cleaning and care agents in an amount of up to about 10 wt. % (based on the total weight of the agent). However, it can also be a component of the aforementioned aqueous-alcoholic carrier.

It was found that the hair treatments agents contemplated herein are also suitable for use as an anti-dandruff preparation. The total weight of anti-dandruff agents based on the total weight of the hair treatment agents can preferably be about 0.01 to about 10 wt. %, more preferably about 0.025 to about 7.5 wt. %, particularly preferably about 0.05 to about 5 wt. %, and in particular about 0.075 to about 3 wt. %. Suitable active anti-dandruff ingredients can be selected from piroctone olamine, climbazole, zinc pyrithione, ketoconazoles, salicylic acid, sulfur, selenium sulfide, tar preparations, undecenoic acid derivatives, burdock root extracts, poplar extracts, stinging nettle extracts, walnut shell extracts, birch extracts, willow bark extracts, rosemary extracts and/or arnica extracts. Climbazole, zinc pyrithione and piroctone olamine are preferred.

Further active ingredients, auxiliary substances and additives that can preferably be present in the hair treatment agents contemplated herein are, for example:
humectants;
perfumes;
UV filters;
thickening agents such as gelatin or gums, for example agar-agar, guar gum, alginates, xanthan gum, gum arabic, karaya gum, locust bean gum, linseed gums, dextrans, cellulose derivatives, for example methyl cellulose, hydroxyalkyl cellulose and carboxymethyl cellulose, starch fractions and derivatives, such as amylose, amylopectin and dextrins, clays and phyllosilicates, such as bentonite or fully synthetic hydrocolloids, such as polyvinyl alcohol, the Ca, Mg or Zn soaps;
structurants such as maleic acid and lactic acid;
dimethyl isosorbide;
cyclodextrins;
fiber structure-improving active ingredients, in particular monosaccharides, disaccharides and oligosaccharides such as glucose, galactose, fructose, fructose and lactose;
dyes for coloring the agent;
active ingredients such as bisabolol and/or allantoin;
complexing agents such as EDTA, NTA, β-alanine diacetic acid, and phosphonic acids;
ceramides. Ceramides shall be understood to mean N-acylsphingosine (fatty acid amides of sphingosine) or synthetic analogs of such lipids (known as pseudoceramides);
propellants such as propane-butane mixtures, N₂O, dimethyl ether, CO₂ and air;
antioxidants;
additional viscosity regulators such as salts (NaCl).
   The agents as contemplated herein are preferably so-called rinse-off products, which is to say they are rinsed off the hair again after a certain exposure time. This exposure time is preferably less than one hour, which is to say the user preferably does not leave the products on the hair until the next time the hair is washed.

A further embodiment contemplated herein is thus a method for treating hair, in which an agent as contemplated herein is applied to dry or moist hair, where it is left for a period of about 30 to about 300 seconds and then rinsed off.

The agents contemplated herein result in considerable greater stability of artificial colorings against washout of the dye. In this way, chemically colored hair may be washed considerably more frequently with the agents contemplated herein, without resulting in undesirable bleeding or fading of the due.

A further embodiment is thus the use of agents contemplated herein to reduce dye from being washed out of chemically colored hair.

What was said above with respect to the agents contemplated herein applies, mutatis mutandis, with respect to preferred embodiments of the method contemplated herein and the use contemplated herein.

### Examples

All information in wt. %

Hair Treatments

| | 1 | 2 | 3 | 4 | 5 | 6 |
|---|---|---|---|---|---|---|
| Cetearyl alcohol | 7.0 | 7.0 | 7.0 | 7.0 | 7.0 | 7.0 |
| Quaternium-87 | 0.9 | 0.9 | 0.9 | 0.9 | 0.9 | 0.9 |
| Cetrimonium chloride | 3.5 | 3.5 | 3.5 | 3.5 | 3.5 | 3.5 |
| Distearoylethyl | 2.0 | 2.0 | 2.0 | 2.0 | 2.0 | 2.0 |
| hydroxyethylmonium | | | | | | |
| methosulfate | | | | | | |
| Polyquaternium-10 | 0.1 | 0.1 | 0.1 | 0.1 | 0.1 | 0.1 |
| Dimethicone | 1.0 | 1.0 | 1.0 | 1.0 | 1.0 | 1.0 |
| Calcium sulfate | 0.8 | 0.8 | 0.8 | 0.8 | 0.8 | 0.8 |
| Baobab seed oil | 0.1 | 0.1 | 0.1 | 0.1 | 0.1 | 0.1 |
| Polyquaternium-37 | 0.4 | 0.4 | 0.4 | 0.4 | 0.4 | 0.4 |
| Hydrolyzed keratin | 0.5 | 0.5 | 0.5 | 0.5 | 0.5 | 0.5 |
| Glycol distearate | 1.0 | 1.0 | 1.0 | 1.0 | 1.0 | 1.0 |
| Citric acid | 1.5 | 1.5 | 1.5 | 1.5 | 1.5 | 1.5 |
| Ethyl lauroyl arginate | 0.08 | 0.08 | 0.08 | 0.08 | 0.08 | 0.08 |
| Dimethyloxazolidine | — | 0.05 | 0.06 | 0.07 | 0.08 | 0.1 |
| Glutaraldehyde | — | 0.05 | 0.06 | 0.07 | 0.08 | 0.1 |
| Hexetidine | — | 0.05 | 0.06 | 0.07 | 0.08 | 0.1 |
| Phenoxyethanol | — | 0.05 | 0.05 | 0.05 | 0.05 | 0.05 |
| Phenoxyisopropanol | — | 1.0 | 0.5 | 0.25 | 0.2 | 0.1 |
| o-cymen-5-ol | — | 0.1 | 0.05 | 0.1 | 0.05 | 0.1 |
| o-phenylphenol | — | 0.2 | 0.1 | 0.2 | 0.1 | 0.1 |
| Propionic acid | — | 0.05 | 0.1 | 0.25 | 0.05 | 0.05 |
| Undecylenic acid | — | 0.1 | 0.2 | 0.1 | 0.2 | 0.1 |
| Dye | 0.2 | 0.2 | 0.2 | 0.2 | 0.2 | 0.2 |
| Perfume | 0.1 | 0.1 | 0.1 | 0.1 | 0.1 | 0.1 |

| | |
|---|---|
| Water | to make 100 |

Hair Rinse Conditioners

| | 1 | 2 | 3 | 4 | 5 | 6 |
|---|---|---|---|---|---|---|
| Cetearyl alcohol | 5.0 | 5.0 | 5.0 | 5.0 | 5.0 | 5.0 |
| Quaternium-87 | 0.75 | 0.75 | 0.75 | 0.75 | 0.75 | 0.75 |
| Glycol distearate | 1.0 | 1.0 | 1.0 | 1.0 | 1.0 | 1.0 |
| Behentrimonium chloride | 1.0 | 1.0 | 1.0 | 1.0 | 1.0 | 1.0 |
| Behenoyl PG trimonium | 1.5 | 1.5 | 1.5 | 1.5 | 1.5 | 1.5 |
| chloride | | | | | | |
| Distearoylethyl | 0.3 | 0.3 | 0.3 | 0.3 | 0.3 | 0.3 |
| hydroxyethylmonium | | | | | | |
| methosulfate | | | | | | |
| Polyquaternium-37 | 0.4 | 0.4 | 0.4 | 0.4 | 0.4 | 0.4 |
| Amodimethicone/ | 0.1 | 0.1 | 0.1 | 0.1 | 0.1 | 0.1 |
| Morpholinomethyl | | | | | | |
| silsesquioxane copolymer | | | | | | |
| Calcium chloride | 0.7 | 0.7 | 0.7 | 0.7 | 0.7 | 0.7 |
| Shea butter | 1.5 | 1.5 | 1.5 | 1.5 | 1.5 | 1.5 |
| Lactic acid | 0.4 | 0.4 | 0.4 | 0.4 | 0.4 | 0.4 |
| Ethyl lauroyl arginate | 1.0 | 1.0 | 1.0 | 1.0 | 1.0 | 1.0 |
| Dimethyloxazolidine | — | 0.4 | 0.3 | 0.2 | 0.1 | 0.05 |
| Glutaraldehyde | — | 0.05 | 0.06 | 0.07 | 0.08 | 0.1 |
| Hexetidine | — | 0.05 | 0.06 | 0.07 | 0.08 | 0.1 |
| Phenoxyethanol | — | 0.05 | 0.05 | 0.05 | 0.05 | 0.05 |
| Phenoxyisopropanol | — | 1.0 | 0.5 | 0.25 | 0.2 | 0.1 |
| o-cymen-5-ol | — | 0.1 | 0.05 | 0.1 | 0.05 | 0.1 |
| o-phenylphenol | — | 0.2 | 0.1 | 0.2 | 0.1 | 0.1 |
| Propionic acid | — | 0.05 | 0.1 | 0.25 | 0.05 | 0.05 |
| Undecylenic acid | — | 0.1 | 0.2 | 0.1 | 0.2 | 0.1 |
| Dye | 0.2 | 0.2 | 0.2 | 0.2 | 0.2 | 0.2 |
| Perfume | 0.1 | 0.1 | 0.1 | 0.1 | 0.1 | 0.1 |

| | |
|---|---|
| Water | to make 100 |

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment. It being understood that various changes may be made in the function and arrangement of elements described in an exemplary embodiment without departing from the scope as set forth in the appended claims.

1. A hair treatment agent, comprising:
at least one fatty alcohol;
at least one cationic surfactant;
at least one divalent or trivalent metal salt;
at least one organic acid; and
ethyl lauroyl arginate.

2. The hair treatment agent according to claim 1, comprising, based on the weight thereof, about 0.1 to about 20 wt. % fatty alcohol(s) of formula (II)
H₃C—(CH₂)ₖ—CH₂—OH (II)
in which k denotes integers from 4 to 28.

3. The hair treatment agent according to claim 1, comprising, based on the weight thereof, about 0.05 to about 20 wt. % cationic surfactant(s).

4. The hair treatment agent according to claim 3, comprising, based on the weight thereof, about 0.05 to about 20 wt. % behenyltrimethylammonium chloride.

5. The hair treatment agent according to claim 1, further comprising, based on the weight thereof, 0.01 to 10 wt. % of at least one divalent or trivalent metal salt from the group of the organic or inorganic copper, zinc, iron(II), calcium, magnesium, iron(III) and/or aluminum salts.

6. The hair treatment agent according to claim 1, comprising, based on the weight thereof, 0.001 to 10 wt. % organic acid(s) from the group consisting of formic acid, acetic acid, propionic acid, butyric acid, isobutyric acid, valeric acid, isovaleric acid, pivalic acid, oxalic acid, malonic acid, succinic acid, glutaric acid, glyceric acid, glyoxylic acid, adipic acid, pimelic acid, suberic acid, azelaic acid, sebacic acid, propiolic acid, crotonic acid, isocrotonic acid, elaidic acid, maleic acid, fumaric acid, muconic acid, citraconic acid, mesaconic acid, camphoric acid, benzoic acid, o,m,p-phthalic acid, naphthoic acid, toluoyl acid, hydratropic acid, atropic acid, cinnamic acid, isonicotinic acid, nicotinic acid, bicarbamic acid, 4,4′-dicyano-6,6′-binicotinic acid, 8-carbamoyloctanoic acid, 1,2,4-pentanetricarboxylic acid, 2-pyrrole carboxylic acid, 1,2,4,6,7-napthalene pentaacetic acid, malonaldehyde acid, 4-hydroxyphthalimide acid, 1-pyrazole carboxylic acid, gallic acid or propanetricarboxylic acid, glycolic acid, lactic acid, malic acid, tartaric acid or citric acid.

7. The hair treatment agent according to claim 6, comprising, based on the weight thereof, 0.001 to 10 wt. % organic acid(s) from the group consisting of formic acid, oxalic acid, maleic acid, lactic acid, tartaric acid or citric acid.

8. The hair treatment agent according to claim 1, comprising, based on the weight thereof, 0.001 to 2 wt. % ethyl lauroyl arginate HCl.

9. A method for treating hair comprising the steps of:
applying to dry or moist hair a hair treatment agent comprising:
at least one fatty alcohol;
at least one cationic surfactant;
at least one divalent or trivalent metal salt;
at least one organic acid; and
ethyl lauroyl arginate, and
rinsing the hair treatment agent from the hair after it is left on the hair for a period of about 30 to 300 seconds.

10. (canceled)

11. The hair treatment agent according to claim 2, comprising, based on the weight thereof, about 2 to about 8 wt. % fatty alcohol(s) of formula (II)
H₃C—(CH₂)ₖ—CH₂—OH (II)
in which k denotes integers from 4 to 28.

12. The hair treatment agent according to claim 2, comprising, based on the weight thereof, 0.1 to 20 wt. % fatty alcohol(s) of formula (II)
H₃C—(CH₂)ₖ—CH₂—OH (II)
in which k denotes integers 10, 12, 14, 16, 18, or 20.

13. The hair treatment agent according to claim 3, comprising, based on the weight thereof, 0.5 to 7 wt. % cationic surfactant(s).

14. The hair treatment agent according to claim 4, characterized by comprising, based on the weight thereof, 0.5 to 7 wt. % behenyltrimethylammonium chloride.

15. The hair treatment agent according to claim 5, further comprising, based on the weight thereof, 0.3 to 3 wt. % of at least one divalent or trivalent metal salt from the group of the organic or inorganic copper, zinc, iron(II), calcium, magnesium, iron(III) and/or aluminum salts.

16. The hair treatment agent according to claim 6, comprising, based on the weight thereof, 0.1 to 2 wt. % organic acid(s) from the group consisting of formic acid, acetic acid, propionic acid, butyric acid, isobutyric acid, valeric acid, isovaleric acid, pivalic acid, oxalic acid, malonic acid, succinic acid, glutaric acid, glyceric acid, glyoxylic acid, adipic acid, pimelic acid, suberic acid, azelaic acid, sebacic acid, propiolic acid, crotonic acid, isocrotonic acid, elaidic acid, maleic acid, fumaric acid, muconic acid, citraconic acid, mesaconic acid, camphoric acid, benzoic acid, o,m,p-phthalic acid, naphthoic acid, toluoyl acid, hydratropic acid, atropic acid, cinnamic acid, isonicotinic acid, nicotinic acid, bicarbamic acid, 4,4′-dicyano-6,6′-binicotinic acid, 8-carbamoyloctanoic acid, 1,2,4-pentanetricarboxylic acid, 2-pyrrole carboxylic acid, 1,2,4,6,7-napthalene pentaacetic acid, malonaldehyde acid, 4-hydroxyphthalimide acid, 1-pyrazole carboxylic acid, gallic acid or propanetricarboxylic acid, glycolic acid, lactic acid, malic acid, tartaric acid or citric acid.

17. The hair treatment agent according to claim 7, comprising, based on the weight thereof, 0.1 to 2 wt. % organic acid(s) from the group consisting of formic acid, oxalic acid, maleic acid, lactic acid, tartaric acid or citric acid.

18. The hair treatment agent according to claim 8, comprising, based on the weight thereof, 0.01 to 1 wt. % ethyl lauroyl arginate HCl.

19. The hair treatment agent according to claim 18, comprising, based on the weight thereof, 0.05 to 0.4 wt. % ethyl lauroyl arginate HCl.

20. The method of claim 9, wherein applying comprises applying the hair treatment agent comprising, based on the weight thereof, 0.001 to 2 wt. % ethyl lauroyl arginate HCl.
